(19) United States
(12) Patent Application Publication
(10) Pub. No.: US 2020/0029832 A1
(43) Pub. Date: Jan. 30, 2020

(54) ABNORMALITY REPORTING DEVICE, RECORDING MEDIUM, AND ABNORMALITY REPORTING METHOD

(71) Applicant: PARAMOUNT BED CO., LTD., Tokyo (JP)

(72) Inventor: Takamasa KOGURE, Tokyo (JP)

(21) Appl. No.: 16/491,740

(22) PCT Filed: Oct. 22, 2018
(86) PCT No.: PCT/JP2018/039211

§ 371 Date: Sep. 6, 2019

(30) Foreign Application Priority Data
Nov. 30, 2017 (JP) 2017-231222

(51) Int. Cl. A61B 5/0245; A61B 5/00; A61B 5/11; A61B 5/08

(52) U.S. Cl. CPC A61B 5/0245; A61B 5/0816; A61B 5/11; A61B 5/4812

(57) ABSTRACT

The present invention is provided with: a biological signal acquisition means for acquiring a biological signal of a user on a bed; a biological information value calculation means for calculating a biological information value from the acquired biological signal; a presuming means for presuming the status of the user on the basis of the biological information value; and a reporting means for reporting an abnormality when the status of the user is determined to be abnormal by the presuming means. As a result, it is possible to provide the abnormality reporting system and the like that can presume the status with high accuracy on the basis of the measured biological information value of the user on a bed.

### TECHNICAL FIELD

The present invention relates to an abnormality reporting device, a recording medium, and an abnormality reporting method.

### BACKGROUND ART

Devices and systems that report an abnormality conventionally have been known. For example, has been know the invention as Patent Literature 1 in which a living activity and a biological activity of a user are detected by a non-invasion type vital sensor and classified into a plurality of classifications, an allowable duration time for each classification is successively cumulated, and when the cumulated time exceeds a threshold value, a report to a caregiver is made.

### CITATION LIST

### Patent Literature

PTL 1: Japanese Patent No. 3557775

### SUMMARY OF INVENTION

### Technical Problem

Conventionally, an abnormality reporting system that reports an abnormality of a user determines whether the user is abnormal or not depending on whether a measurement value (biological information value) related to the abnormality of the user has exceeded a threshold value. The abnormality reporting system generally reports to a staff such as a health care worker or a caregiver, on the basis of the determination result as to whether the user is abnormal. For example, in the above-mentioned invention described in Patent Literature 1, regardless of the status of the user and the degree of relation to the abnormality, when the cumulated time of the living activity and the biological activity exceeds a prescribed threshold value, an abnormality is determined, and a report to the caregiver is made on the basis of the determination result. The accuracy of determining an abnormality is lowered depending on the status of the user, and the degree of relation between the living activity or the biological activity and the abnormality varies. Simply determining an abnormality only on the basis of the biological information value of the user, regardless of the status of the user and the like, causes a report with no reliability in some cases.

The conventional system that reports an abnormality of the user sets a normal range of a biological information value such as a heart rate and a respiratory rate highly related to the status of the user, and an abnormality of the user is determined when the biological information value has exceeded the normal range. In this case, the system misses an abnormality when the biological information value does not deviate from normal range.

The system erroneously determines an abnormality when the heart rate of the user has temporarily risen by exercise. When the user is an athlete having a high cardiopulmonary function and has a low heart rate even in normal times, the system erroneously determines an abnormality in some cases. Similarly, the heart rate of some users does not deviate from the normal range even in abnormal times, and the system cannot determine an abnormality in some cases.

The conventional system has determined an abnormality only on the basis of the biological information value of the user being deviated from the normal range. When the system reports on the abnormality of the user to a caregiver or the like, many incorrect reports and undetected errors of the abnormality have easily occurred in some cases.

There is a method in which, in order to prevent an influence by a transient abnormality value due to mixing of noise and an artifact, which is not related to the abnormality, in differences among individuals of the users and the detected biological signals of the user, a status of a patient for a prolonged period of time and the change in data such as the biological signal value are analyzed to determine an abnormality. In the method of grasping the change in the data for a prolonged period of time, the conditions for acquiring data need to be uniformed and an analysis is performed on the basis of the data under the same condition. For example, when data includes data when the user exercises and data when the user is at rest are mixed, the accuracy to determine an abnormality is lowered if the change in the both data is analyzed in a separated manner.

Specifically, in a case of a system that is used in a hospital and a care facility and reports an abnormality on the basis of a biological information value, an unnecessary abnormality report based on an error and the like brings an unnecessary confirmation transaction and causes burden, to a health care worker and a staff. When the system misses an abnormality of the user, a fatal situation occurs.

In view of the above-mentioned problems, an object of the present invention is to provide an abnormality reporting device and the like capable of presuming a status of a user with high accuracy on the basis of a biological information value of the user.

### Solution to Problem

In order to solve the above-mentioned problems, an abnormality reporting device according to one aspect of the present invention is characterized by including: a biological signal acquisition unit that acquires a biological signal of a user on a bed; a biological information value calculation unit that calculates a plurality of types of biological information values from the acquired biological signal; a presumption unit that presumes a status of the user on the basis of the plurality of types of the biological information values; and a report unit that reports when the status of the user has been determined to be abnormal by the presumption unit.

A computer-readable recording medium according to another aspect of the present invention records therein a program for causing a computer to execute processing, the processing including: a step of acquiring a biological signal of a user on a bed; a step of calculating a plurality of types of biological information values from the acquired biological signal; a step of presuming a status of the user on the basis of the plurality of types of the biological information values; and a step of reporting when the status of the user has been determined to be abnormal by the presumption function.

An abnormality reporting method according to still another aspect of the present invention in an abnormality reporting device capable of reporting an abnormality when a status of a user being abnormal is determined, is characterized by including: an biological signal acquisition step of acquiring a biological signal of the user on a bed, by the abnormality reporting device; a biological information calculation step of calculating a plurality of types of biological information values from the acquired biological signal, by the abnormality reporting device; a presumption step of presuming a status of the user on the basis of the plurality of types of the biological information values, by the abnormality reporting device; and a report step of reporting when the status of the user has been determined to be abnormal at the presumption step, by the abnormality reporting device.

### Advantage of the Invention

A biological information value is calculated from a biological signal of a user on a bed, and a status of the user is presumed on the basis of the biological information value. When the status of the user is determined to be abnormal, a report is made. By using biological information when the user is lying down, it is possible to presume a status of the user appropriately, and to report on the abnormality.

### BRIEF DESCRIPTION OF THE DRAWINGS

FIG. 1 is a diagram for explaining the whole in a first embodiment.

FIG. 2 is a diagram for explaining a functional configuration in the first embodiment.

FIG. 3 is an operation flow for explaining patient presumption processing in the first embodiment.

FIG. 4 is a diagram for explaining a functional configuration of a patient presumption unit in a second embodiment.

FIG. 5 is a diagram for explaining a functional configuration in a third embodiment.

FIG. 6 is a diagram for explaining a neural network in the third embodiment.

FIG. 7 is a diagram for explaining a patient diary (diary data) for explaining an example.

FIG. 8 is a diagram for explaining a patient diary (sleep diary) for explaining the example.

FIG. 9 is a diagram for explaining a patient diary (respiratory diary) for explaining the example.

FIG. 10 is a diagram for explaining a patient diary (heartbeat diary) for explaining the example.

FIG. 11 is a diagram for explaining a patient diary (respiratory event diary) for explaining the example.

FIG. 12 is a diagram for explaining a patient diary (cyclic body movement diary) for explaining the example.

FIG. 13A is a diagram for explaining a patient diary (diary data) for explaining the example.

FIG. 13B is a diagram for explaining a patient diary (diary data) for explaining the example.

FIG. 13C is a diagram for explaining a patient diary (diary data) for explaining the example.

FIG. 14A is a diagram for explaining a patient diary (diary data) for explaining the example.

FIG. 14B is a diagram for explaining a patient diary (diary data) for explaining the example.

FIG. 14C is a diagram for explaining a patient diary (diary data) for explaining the example.

### DESCRIPTION OF EMBODIMENTS

Hereinafter, embodiments discussed herein are described with reference to the drawings. Specifically, a case where an abnormality reporting device in the present invention is applied will be described, however, the range in which the present invention is applied is not limited to the embodiments.

### 1. First Embodiment

[1.1 Overall System]

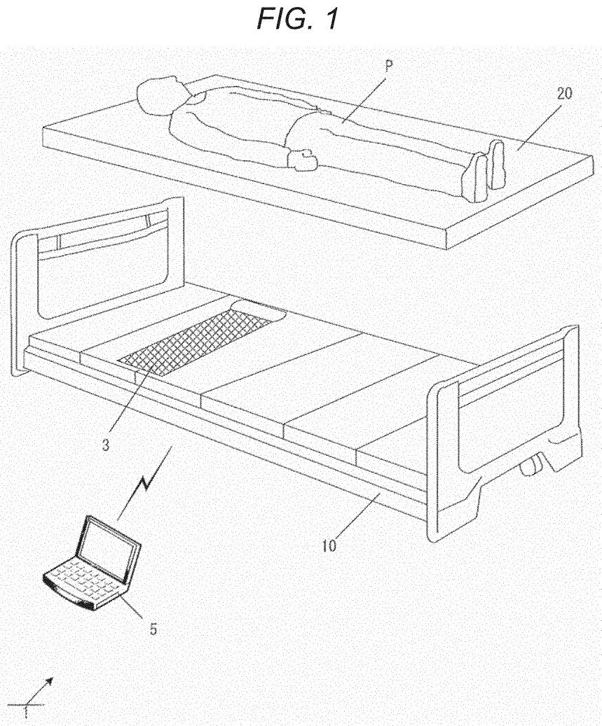

FIG. 1 a diagram for explaining an overall overview of a system 1 to which the present invention is applied. As illustrated in FIG. 1, the system 1 includes a detection device 3 that is placed between a bed portion of a bed 10 and a mattress 20, and a processor 5 for processing a value output from the detection device 3. The detection device 3 and the processor 5 constitute an output device of biological information.

When a user P is present on the mattress 20, the detection device 3 detects, as a biological signal of the user P, body vibration (vibration emitted from a human body). The detection device 3 calculates, on the basis of the detected vibration, a biological information value of the user P. In the present embodiment, the detection device 3 can output the calculated biological information values (at least, the respiratory rate, the heart rate, and the amount of activity), as biological information values of the user P. The detection device 3 may detect vibration, and the processor 5 may calculate a biological information value. The processor 5 can output and display the biological information value.

The detection device 3 and the processor 5 may be integrally configured by providing a memory, a display unit, and the like to the detection device 3. The processor 5 may be a general device, and thus is not limited to an information processor such as a computer, but may include a device, for example, a tablet computer or a smartphone.

The user is a user of a bed device, and may be a person (patient) under medical treatment of a disease or a person (care receiver) who needs care. The user may be a healthy person who needs no care, an elderly person, a child, a handicapped person, or also an animal not a person.

The detection device 3 is formed in a sheet shape so as to have a thin thickness. This enables the user P without having a discomfort feeling to use the detection device 3 even when being placed between the bed 10 and the mattress 20. This enables the detection device 3 to measure a biological information value on the bed for a prolonged period of time (for example, measurement for a predetermined period of time such as one hour or more and eight hours or more, and a predetermined period of time such as one night, one sleep, one week, one month, one year, and ten years or more). The detection device 3 calculates a biological information value from the body vibration, and thus cannot measure a respiratory rate and a heart rate when the user moves his/her body (the measurement accuracy in the respiratory rate and the heart rate is lowered when the body is moved, which causes noise in the abnormality reporting system). The detection device 3 acquires a biological information value and the like in a status of a patient, which is limited to a resting status. The detection device 3 further has a function of determining the reliability in the measured body vibration data, and recording only the data with high reliability.

As the methods of calculating biological information values of the user, for example, the sleeping status determination methods described in JP-A 2010-264193 (Title of the Invention: SLEEPING CONDITION DECISION INSTRUMENT, PROGRAM, AND SLEEPING CONDITION DECISION SYSTEM, filed on May 18, 2009) and JP-A 2015-12948 (Title of the Invention: SLEEP EVALUATION DEVICE, SLEEP EVALUATION METHOD, AND SLEEP EVALUATION PROGRAM, failed on Jul. 4, 2013) can be referred. The entire contents of these patent applications are incorporated herein by reference.

The detection device 3 only needs to acquire a biological signal (signal of body movement, respiratory motion, ballistocardiographic movement, or the like) of the user P. In the present embodiment, the detection device 3 calculates a heart rate and a respiratory rate on the basis of the body vibration. The detection device 3 may, in addition to the above, for example, acquire a biological signal of the user using an infrared ray sensor, acquire a biological signal of the user using the acquired video, or acquire a biological signal using an actuator with a strain gauge. The detection device 3 may be implemented by a smartphone, a tablet computer, or the like. In this case, the detection device 3 acquires a biological signal using an acceleration sensor or the like that is incorporated in the smartphone and the tablet computer.

Moreover, “bed” is a place where a patient that is a user sleeps. The bed normally indicates a portion on a section of the bed device or on a mattress that is placed on the section, on a portion on an air cell, on a portion on a bedding, and other portions. Moreover, it is assumed that the bed widely includes a seat for an automobile and a sofa, as long as a place where the patient sleeps.

[1.2 Functional Configuration]

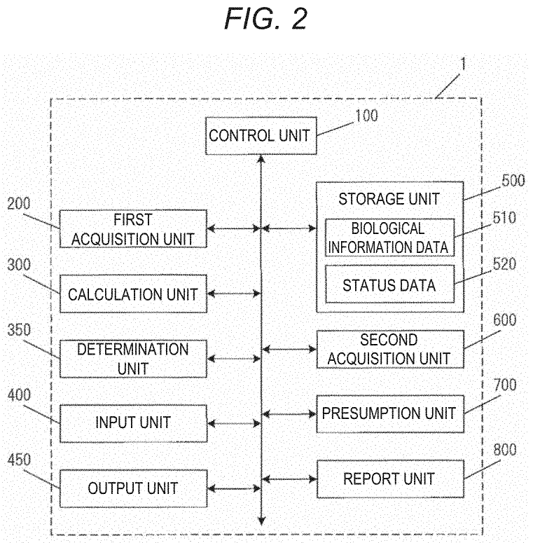

A functional configuration of the system 1 will be described using FIG. 2. The system 1 in the present embodiment includes the detection device 3 and the processor 5, and respective function units (processes), other than a first acquisition unit 200, may be implemented by either one of the devices. These devices are combined to function as an abnormality reporting device.

The destination to which the system 1 reports is a health care worker, a staff in a care facility, a family of the user, when the status of the user is abnormal. These health care worker, staff in the care facility, family of the user, and the like are collectively called a staff. As a method that the system 1 reports to the staff on a status of the user, the system 1 may simply report by means of sounds and a screen display, or may report to a mobile terminal device by means of email and the like. The system 1 may report to a terminal device or the like, other than that that is connected to the detection device 3.

The system 1 (abnormality reporting device) includes a control unit 100, the first acquisition unit 200, a calculation unit 300, a determination unit 350, an input unit 400, an output unit 450, a memory 500, a second acquisition unit 600, a presumption unit 700, and a report unit 800. In the case of FIG. 1, the detection device 3 includes the control unit 100, the first acquisition unit 200, and the memory 500, and the processor 5 includes the units other than those units. The second acquisition unit 600 may be implemented using the first acquisition unit 200, or may be separately provided to the bed 10.

The control unit 100 is a function unit for controlling an operation of the system 1. For example, the control unit 100 may include a control device such as a central processing unit (CPU), or may include a control device such as a computer. The control unit 100 reads and executes various kinds of programs stored in the memory 500, thereby implementing various kinds of processes. The control unit 100 may be provided to each of the detection device 3 and the processor 5.

The first acquisition unit 200 acquire a biological signal of the user. In the present embodiment, the first acquisition unit 200 detects a body vibration that is one type of a biological signal, using a sensor that detects a change in pressure, as one example. The first acquisition unit 200 acquires a biological signal such as the respiratory and the heartbeats from the detected body vibration of the user. The control unit 100 and the calculation unit 300 each convert the biological signal into biological information value data such as the respiratory rate, the heart rate, the amount of activity. The control unit 100 can acquire and determine a status of the user on the basis of the detected body vibration. The status of the user indicates which status the user is in. For example, the status of the user indicates whether the user is present on the bed or is absent from the bed, a posture (for example, sitting-on-bed-edge and the like) of the user, a position on the bed device, whether the user is in a sleeping status or an awake status, and a status (sleeping status) of the user when sleeping.

The first acquisition unit 200 in the present embodiment detects a body vibration of a patient by a pressure sensor, for example, and acquires a biological signal of the respiratory, the heartbeat, or the like from the body vibration. The first acquisition unit 200 may acquire a biological signal by a load sensor, from changes in the center of gravity position of the patient and the load value, or may acquire a biological signal, by providing a microphone, on the basis of the sounds picked up by the microphone. The first acquisition unit 200 only needs to be capable of acquiring a biological signal of the patient using any of the sensors.

The first acquisition unit 200 may be provided to the detection device 3, or may receive a biological signal from an external device.

The calculation unit 300 calculates biological information values of the user P (the respiratory rate, the heart rate, and the like). In the present embodiment, the calculation unit 300 can extract a respiratory component and a heartbeat component from the body vibration acquired by the first acquisition unit 200, and calculate biological information values of the respiratory rate and the heart rate on the basis of the respiratory interval and the heartbeat interval. The calculation unit 300 may analyze (Fourier transform and the like) the periodicity of the body vibration, and calculate, from the peak frequency, biological information values such as a respiratory rate and a heart rate, or may calculate a biological information value using the pattern recognition and the artificial intelligence (machine learning). As a calculation method of a respiratory rate and a heart rate, for example, the method disclosed in Journal of Japanese Society of Sleep Research whose title is Sleep evaluation by a newly developed PVDF sensor non-contact sheet: a comparison with standard polysomnography and wristactigraphy written by Sunao UCHIDA, Takuro ENDO, Kazue SUENAGA, Hideto IWAMI, Shinsuke INOUE, Eiji FUJIOKA, Ayako IMAMURA, Takafumi ATSUMI, Yoshitaka INAGAKI and Atsushi KAMEI, published in 2011, can be referred and incorporated herein.

The determination unit 350 determines a status of the user when being present on the bed. For example, the determination unit 350 determines, on the basis of the biological signal (body vibration) acquired by the first acquisition unit 200, whether the user is awake or is sleeping. The determination unit 350 may determine a sleeping status of the user as “REM sleep” or “non-REM sleep”, and may determine a depth of the sleep.

As the methods of determining a sleeping status of the user, for example, the sleeping status determination methods described in JP-A 2010-264193 (Title of the Invention: SLEEPING CONDITION DECISION INSTRUMENT, PROGRAM, AND SLEEPING CONDITION DECISION SYSTEM, filed on May 18, 2009) and JP-A 2016-87355 (Title of the Invention: SLEEP STATE DETERMINATION DEVICE, AND SLEEP STATE DETERMINATION METHOD AND PROGRAM, filed on Nov. 11, 2014) can be referred. The entire contents of these patent applications are incorporated herein by reference.

With the input unit 400, a user or a staff inputs various conditions, and performs a manipulation input of the measurement start. For example, the input unit 400 is implemented by any input means including a hardware key and a software key.

The output unit 450 outputs various information. The output unit 450 outputs, for example, a biological information value such as a heart rate or a respiratory rate, and a status of the user. The output unit 450 may output an abnormality being occurred when the status of the user is abnormal. The output unit 450 may be a display device such as a display and the like, or may be a sound output device that outputs a warning and the like. The output unit 450 may be an external storage device that outputs and stores a status of the user and a biological information value, or may be a transmission device or a communication device that transmits a status of the user and a biological information value to another device.

The memory 500 stores therein various kinds of data and programs with which the system 1 operates. The control unit 100 implements the function by reading and executing the program stored in the memory 500. The memory 500 includes, for example, a semiconductor memory, a magnetic disk device, and the like. The memory 500 stores therein biological information data 510 and status data 520.

The biological information data 510 stores therein a biological signal of the user (body vibration data), and biological information values (a respiratory rate, a heart rate, and the like) to be calculated from the biological signal. The biological information data 510 stores therein a respiratory rate, a heart rate, and body vibration data, but only needs to store therein at least one among the rates and the data if necessary. The biological information data 510 may further store therein other information (for example, an respiratory event index based on the variation and the like of the respiratory amplitude, and a periodic body movement index based on the periodicity of the body movement), as long as other information is a biological information value that can be calculated by the calculation unit 300.

The status data 520 stores therein a status of a user. For example, the status data 520 stores therein a status of the user (first status) output by the determination unit 350, and a status of the user (second status) output by the second acquisition unit 600.

The status data 520 stores therein whether a user is an awake status or a sleeping status, as the first status of the user. The status data 520 may further store therein a status such as a REM sleep or a non-REM sleep, as the sleeping status of the user. The status data 520 stores therein whether the user is present on the bed or is absent from the bed, as a second status of the user. The status data 520 may store therein, when the user is present on the bed, a posture of the user and a position thereof on the bed device (for example, the bottom or the mattress).

The second acquisition unit 600 acquires a second status of a user. For example, the second acquisition unit 600 acquires whether the user is absent from the bed or is present on the bed, that is a present-in-bed status of the user, by the first acquisition unit 200 or a load sensor or the like that is separately provided from the first acquisition unit 200. When the user is present on the bed, the second acquisition unit 600 may acquire a position (for example, whether the user is in a sitting-on-bed-edge) or a posture (for example, whither the user is in a supine position) of the user on the bed device. The second acquisition unit 600 may acquire a sitting posture or a sleeping posture, as the posture.

The presumption unit 700 presumes whether the user is in an abnormal status (a third status of the user), from various parameters such as the biological signal, the biological information value, the first status of the user determined by the determination unit 350, and the second status of the user acquired by the second acquisition unit 600. When the presumption unit 700 presumes a status of the user to be abnormal, the report unit 800 outputs (reports) an alert.

As the timing when the presumption unit 700 may presume a third status of the user, the presumption unit 700 may presume in real-time or at every interval of predetermined time. As the timing when the presumption unit 700 presumes a status of the user, for example, the presumption unit 700 may presume for every five minutes or may presume for every one hour. The presumption unit 700 may periodically presume a status of the user once at the fixed time in the morning or at night (for example, six o'clock in the morning, nine o'clock at night, or the like), or may presume a status of the user two times or three times at the fixed times. The presumption unit 700 may presume a third status of the user when the user falls asleep, at the timing when 30 minutes has passed since the user has been present on the bed.

The presumption unit 700 presumes a third status of the user at the fixed time once every day. The presumption unit 700 can presume under the same condition, and obtain an effect of an increased presumption accuracy. In particular, presuming a third status of the user at the fixed time once every day when the user wakes up effectively reduces an incorrect report and an undetected report. A status being abnormal as a third status of the user mainly appears in an biological information value that is calculated while the user is sleeping. The presumption unit 700 compares information in a period of time from when the user goes to bed to when the user wakes up on that day (or a certain time zone during the night) with past information, and evaluates a time-elapsed change in the biological information value in a period of time from when the user goes to bed to when the user wakes up on that day (or a certain time zone during the night), which are effective for presuming an abnormality.

For example, the system 1 erroneously reports on an abnormality at 3:00 at midnight to cause a large adverse influence from both sides of the labor of a person who responds to the report and the disturbance in sleep of the user due to the response. At around the time when the user wakes up every morning, a person to be responded (a nurse or a caregiver) can additionally confirm a status when prompting the user (patient or person in need of nursing care) to wake up. For the user, it is important to wake up every morning at regular time for securing the regularity of the biological rhythm, which results in improvement in the degree of arousal during the daytime and good-quality sleeping during the night.

[1.2 Flow of Processing]

A method in which the presumption unit 700 presumes an abnormality as a status of a user in the present embodiment will be described.

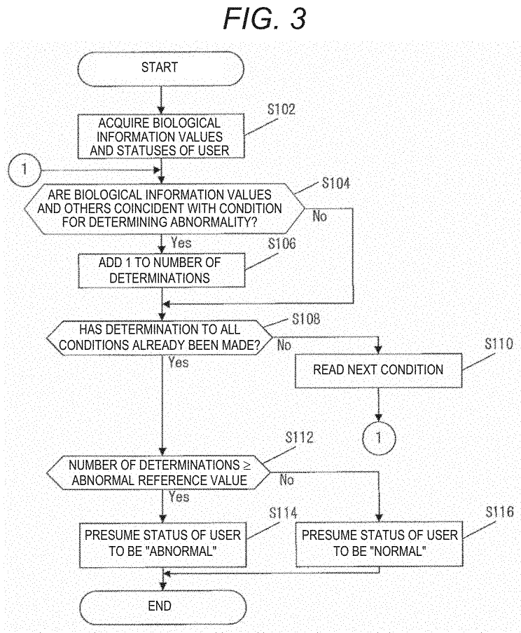

FIG. 3 is an operation flow for explaining presumption processing of presuming a status of a user. In the present embodiment, the presumption unit 700 presumes a status of a user by executing the processing in FIG. 3.

Firstly, the presumption unit 700 acquires (calculates) biological information values and statuses of the user (first status and second status) (Step S102). As for the biological information values to be acquired by the presumption unit 700, the respiratory rate, the heart rate, and the amount of activity are important. The presumption unit 700 acquires a parameter to be used for a condition that determines an abnormality, which is described later. For example, the presumption unit 700 acquires statuses of the user (first status and second status) such as sleeping or wakening of the user (presence-on-bed), absence-from-bed, and the like, if necessary. This enables the presumption unit 700 to presume a more detailed status of the user, by taking such changes that the user has become difficult to sleep, has stayed on the bed for an increased period of time, has left the bed for an increased period of time, and other changes, a continuous presence-on-bed time, a continuous absence-from-bed time, and the like, into consideration.

The presumption unit 700 may further use, as one of the biological information values, an index (biological index) related to the user. The biological index includes a respiratory event index, a periodic body movement index, and the like. The presumption unit 700 can presume, by acquiring biological information values, statuses of the user and/or biological indices (hereinafter, biological information values and others), a more detailed status of the user, from absolute values of the biological information values and others, a change in daily average value, a change in time-series distribution for 24 hours, and the like. The presumption unit 700 may acquire and use a history of the biological information values and others to acquire a past value, an average value, a standard deviation, a variation coefficient, and a value and a ratio of a change for the latest predetermined period of time, and may presume a status of the user.

The presumption unit 700 may acquire biological information values from the calculation unit 300 as biological information values, or may calculate biological information values by acquiring a biological signal from the first acquisition unit 200 and executing a prescribed computation. The biological information value and the index may be respectively calculated from different one or a plurality of biological information values.

Subsequently, the presumption unit 700 determines whether the biological information values and others are coincident with a condition for determining an abnormality (Step S104). If the biological information values and others are coincident with the condition, the presumption unit 700 adds 1 to the number of determinations for determining an abnormality (Step S104; Yes→Step S106). If the determination with respect to all the conditions for determining an abnormality has not been finished, the presumption unit 700 reads a next condition for determining an abnormality. The presumption unit 700 then determines whether the biological information values and others are coincident with the condition for determining an abnormality (Step S108; No→Step S110→Step S104).

When the presumption unit 700 presumes a third status of the user, the presumption unit 700 determines whether the biological information values and others are coincident with one or a plurality of conditions for determining an abnormality, on the basis of the biological information values and others, and the statuses of the user (first status and second status). One example of a condition for determining an abnormality will be described below.

As a condition depending on which the presumption unit 700 determines an abnormality, the following conditions can be considered. The biological information value acquired at the timing below is used in each condition.

Normally, the presumption unit 700 uses, among the biological information values of the user acquired on the bed, as the heart rate, the respiratory rate, and the amount of activity, the biological information values in a time zone with less noise, in other words, during the night rather than during the daytime, the time when the user is sleeping rather than the time when the user is awake.

When a user is bedridden with less noise even during the daytime, the presumption unit 700 may use data other than data during the night. When the physical condition of the user becomes worse, a change in biological information value appears in a status of absence-from-bed and presence-on-bed for 24 hours, which includes cases where the presence-on-bed time increases during the daytime, the absence-from-bed increases due to restlessness, the user wakes up in the early morning. The presumption unit 700 uses the biological information values to the condition, irrelevant to whether the time is during the daytime or during the night, the time when the user is awake or the time when the user is sleeping.

Hereinafter, the conditions are enumerated.

(1) An average respiratory rate for the latest 30 minutes (not an instantaneous value, but a value for comparatively a long period of time is used to obtain excellent accuracy).

(2) A difference between the latest and past average values of the average respiratory rate during the night (the variation in an average respiratory rate during the night varies small in a person, and the accuracy is excellent).

(3) An average heart rate for the latest 60 minutes (the measurement accuracy thereof is lower than that of the respiratory rate, and thus a calculation time longer than that in (1) is set).

(4) A difference between the latest and past average values of the average heart rate during the night (the measurement accuracy thereof is lower than that of the respiratory rate, and thus the abnormality determination condition is set to be more difficult to be satisfied than (2) or a small weight is assigned to the abnormality determination result).

(5) An inclination of a linear approximation straight line of the respiratory rate during the night (the accuracy is high due to the comprehensive variation tendency. an index capable of evaluating whether the respiratory rate from the night to the morning is in an upward trend or a downward trend, such as a difference between an average value in the first half during the night and an average value in the second half during the night may be used).

(6) An inclination of a linear approximation straight line of the heart rate during the night (the accuracy is high due to the comprehensive variation tendency, but the accuracy is lower than that of the respiratory rate, and thus the abnormality determination condition is set to be more difficult to be satisfied, or a small weight is assigned to the abnormality determination result. an index capable of evaluating whether the heart rate from the night to the morning is in an upward trend or a downward trend, such as a difference between an average value in the first half during the night and an average value in the second half during the night may be used).

(7) A fluctuation of the respiratory rate during the night (individually unique index, and the accuracy is high due to the comprehensive variation tendency. a standard deviation, a variation coefficient, or the like.).

(8) A fluctuation of heart rate during the night (individually unique index, the accuracy is high due to the comprehensive variation tendency, but the accuracy is lower than that of respiratory rate, and thus the abnormality determination condition is set to be difficult to be satisfied, or a small weight is assigned to the abnormality determination result. a standard deviation, a variation coefficient, or the like).

(9) A difference between the latest and past average values of the average amount of activity during the night (an individually unique index, the accuracy is high due to the comprehensive variation tendency).

(10) A difference between the latest and past average values of the average respiratory event index during the night (individually unique index, the accuracy is high due to the comprehensive variation tendency).

(11) A difference between the latest and past average values of the average periodic body movement index during the night (individually unique index, the accuracy is high due to the comprehensive variation tendency).

(12) A difference between the latest and past average values of the average absence-from-bed time during the night (individually unique index, the accuracy is high due to the comprehensive variation tendency).

(13) A cumulated value (cumulated value: 0 to 1440 for every one minute) of a difference between an average presence-on-bed rate (0 to 1) for 24 hours (for every one minute) and a determination (presence-on-bed: 1, absence-from-bed: 0) for the latest 24 hours (individually unique index, the accuracy is high due to the comprehensive variation tendency).

(14) An average amount of activity for the latest eight hours (an index highly related to the activity property, the accuracy is high due to the comprehensive variation tendency).

The presumption unit 700 determines whether the biological information values and others exceed a reference value on the basis of each condition. For example, in a case of the condition (1), the presumption unit 700 uses the respiratory rate among the inputted biological information values, and determines whether the respiratory rate is coincident with the condition for determining an abnormality. For example, the presumption unit 700 calculates an average respiratory rate for the latest 30 minutes. When the calculated average respiratory rate does not fall within a reference value (for example, 8 to 28), the presumption unit 700 adds 1 to the number of determinations.

The presumption unit 700 may change the method of determining whether the status of the user is abnormal. The presumption unit 700 changes the condition as appropriate depending on an attribution, a current disease, and a medical history of the user, the characteristic of the first acquisition 200, whether less incorrect report or less overlooking (undetected report) is intended, and the like. For example, when the condition is changed depending on the medical history of the user, the presumption unit 700 may raise the degree of importance of a corresponding condition when the user has a chronic disease in the heart and attention needs to be paid. The presumption unit 700 may lower the degree of importance of the condition related to the heart rate because the accuracy of the heart rate is dropped when the user has a chronic disease in the heart and suffers from arrhythmia.

For example, when the first acquisition unit 200 has a difference between accuracies as a characteristic thereof, it can be considered that the weight to the condition is changed in accordance with the characteristic of the first acquisition unit 200. For example, when the first acquisition unit 200 is placed under the user and detects the body vibration, the presumption unit 700 can acquire the respiratory rate more accurately than the heart rate. As a condition that the presumption unit 700 determines an abnormality, the respiratory rate is weighted heavier.

When the first acquisition unit 200 is an electrocardiograph, the presumption unit 700 can acquire the heart rate more accurately than the respiratory rate. As a condition that the presumption unit 700 determines an abnormality, the heart rate is weighted heavier. In accordance with the type and the characteristic of the first acquisition unit 200 (sensor), the presumption unit 700 may assign the degree of importance (weighting and priority) of the condition when a status of the user is determined.

It is important for the presumption unit 700 to combine these conditions in plurality and presume a status of the user. For example, when “3” is set as an abnormality reference value, the presumption unit 700 presumes the status of the user (third status) to be “abnormal” if the number of determinations is “3” that is the abnormality reference value or more (Step S112; Yes→Step S114). If the number of determinations is less than the abnormality reference value, the presumption unit 700 presumes a status of the user (third status) to be normal. For example, when the number of determinations is “2” or less (when the abnormality reference value is “3”), the presumption unit 700 presumes the status of the user to be normal.

In FIG. 3, the presumption unit 700 uses the number of determinations and the abnormality reference value, and presumes a status of the user with the number of determinations determined to be abnormal under the condition for determining an abnormality. The presumption unit 700 can presume a status of the user by another method.

For example, in place of determining a coincidence with each condition for determining an abnormality, the presumption unit 700 calculates the degree of abnormality from a determination formula that determines the degree of abnormality for each abnormality. The presumption unit 700 may presume a status of the user using a total value of the calculated degrees of abnormality. For example, the presumption unit 700 sets the degree of abnormality that is calculated for each condition as an explanatory variable and the overall degree of abnormality as an objective variable, and executes a multivariate analysis (multi regression analysis and the like). The presumption unit 700 may presume a status of the user from the calculated overall degree of abnormality.

The presumption unit 700 does not need to use all the conditions for determining an abnormality, and may use the conditions in combination if necessary. The strength of relation between each condition for determining an abnormality and the real abnormality is not uniform. The presumption unit 700 may weight the number of determinations for determining an abnormality depending on an attribution, a current disease and a medical history of the user, the characteristic of the first acquisition unit 200, whether less incorrect report or less overlooking (undetected report) is intended, and the like. The presumption unit 700 may weight the number of determinations in accordance with the strength of relation with the real abnormality, and may presume a status of the user using the weighted number of determinations.

The presumption unit 700 may presume a status of the user using, among a plurality of conditions for determining an abnormality, the important condition with priority. For example, when the first acquisition unit 200 is placed under the user and is based on the body vibration, among the above-mentioned conditions, the condition (1) has the highest effect. The presumption unit 700 may presume a status of the user by using the condition (1) with priority, and weight the condition (1) as being important.

### 2. Second Embodiment

A second embodiment will be described. In the first embodiment, the explanation in which the presumption unit 700 determines the inputted biological information on the basis of the condition for determining an abnormality, and presumes a status of a user has been made.

In the present embodiment, a case in which the presumption unit 700 uses artificial intelligence (machine learning), and presumes a status of the user (third status) will be described.

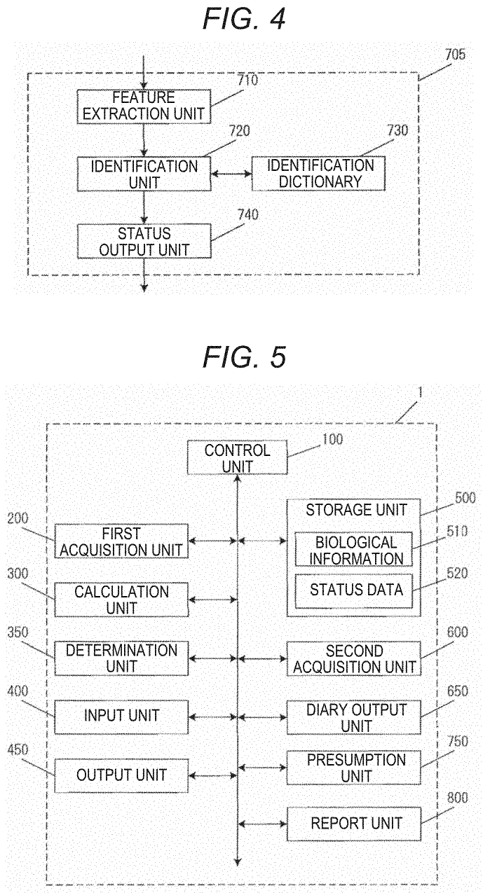

In the present embodiment, in place of the presumption processing in FIG. 3, a status of the user is presumed on the basis of a presumption unit 705 in FIG. 4.

An operation of the presumption unit 705 in the present embodiment will be described. The presumption unit 705 uses a biological information value and a status of the user as an input value (input data), and presumes a status of the user using artificial intelligence and various kinds of statistical indices.

As illustrated in FIG. 4, the presumption unit 705 includes a feature extraction unit 710, an identification unit 720, an identification dictionary 730, and a status output unit 740.

The presumption unit 705 inputs and uses various parameters. As the parameter, for example, in the present embodiment, on the basis of the body vibration data acquired from the first acquisition unit 200, the presumption unit 705 uses biological information values calculated by the calculation unit 300 and statuses of the user (first status and second status). The presumption unit 705 uses, as the biological information values, for example, the “respiratory rate”, the “heart rate”, and the “amount of activity”. As the biological information values, the “fluctuation of respiratory rate” and the “fluctuation of heart rate” calculated from these biological information values, and the “respiratory event index” and the “periodic body movement index” calculated from the same body vibration data are also available.

The presumption unit 705 can use, as the statuses of the user (first status and second status), a status whether the user is present on the bed or is absent from the bed, as a present-in-bed status of the user. When the user is present on the bed, the presumption unit 705 can use a status whether the user is in an awake status or is in a sleeping status. When the user is in a sleeping status, the presumption unit 705 may use a REM sleep/non-REM sleep, or a depth of the sleep of the user.

The presumption unit 705 uses the number of significant variations of the respiratory amplitude per one hour of sleep as the “respiratory event index”. The presumption unit 705 may use the number of times of apnea per one hour of sleep (apnea index) or the total number of times of apnea and hypopnea per one hour of sleep (apnea hypopnea index) in some cases. The presumption unit 705 uses the number of times of generation of periodical body movement per one hour of sleep as the “periodic body movement index”. The presumption unit 705 may use the number of times of periodic limb movement per one hour of sleep.

The feature extraction unit 710 extracts a feature point on the basis of the inputted parameter, and outputs the feature point as a feature vector. The feature points to be extracted by the feature extraction unit 710 can be considered as follows, for example.

(1) The respiratory rate of 30 [times/min] or more or 8 [times/min] or less continues for a certain period of time or more.

(2) The heart rate of 120 [times/min] or more or 40 [times/min] or less continues for a certain period of time or more.

(3) The trend of the heart rate or the respiratory rate from the start to the end of the night sleep rises (10% or more).

(4) The fluctuation of the respiratory rate or the heart rate (standard deviation, variation coefficient) during the night (21:00 to 6:59) is a constant value or more.

(5) The respiratory event index or the periodic body movement index significantly decreases.

(6) The respiratory event index or the periodic body movement index significantly increases, or is a constant value or more (during the night).

(7) The amount of activity significantly increases or decreases.

(8) The sleep determination continues for a certain period of time or more, and the wakening determination during the night is 95% or more.

The feature extraction unit 710 combines one or a plurality of these feature points, thereby outputting a feature vector. The feature points having been described are only examples, and the values are not limited thereto. If considering (1) as an example, the condition of (1) may be the respiratory rate of 25 [times/min] or more or may be the respiratory rate of 10 [times/min] or less. The respective values are used for convenience of explanation as just described. The feature extraction unit 710 may output the corresponded feature point as “1” and the non-corresponded feature point as “0”, or may output a random variable.

When all the above-mentioned feature points are included, the feature space is an eight-dimensional space, and the feature extraction unit 710 outputs the feature points as an eight-dimensional feature vector to the identification unit 720.

The identification unit 720 identifies a class corresponding to the status of the user from the inputted feature vector. In this process, the identification unit 720 verifies the inputted feature vector against a plurality of prototypes prepared in advance as the identification dictionary 730, thereby identifying a class. The prototype may be stored as a feature vector corresponding to each class, or may be stored as a feature vector representative of the class.

When the identification dictionary 730 stores therein feature vectors representative of the classes, the identification unit 720 determines a class to which the nearest prototype belongs. The identification unit 720 may determine a class by the nearest neighbor rule, or may determine a class by the k-nearest neighbor algorithm.

The identification dictionary 730 that the identification unit 720 uses may store therein in advance a prototype, or may store therein using the machine learning.

The status output unit 740 then outputs a status of the user (third status) corresponding the class identified by the identification unit 720. The status output unit 740 outputs “normal” or “abnormal” as the status of the user (third status). When the status of the user is abnormal, the status output unit 740 may further output a status such as “fever” or “condition change”. The status output unit 740 may output a random variable. The status of the user output by the status output unit 740 is the status of the user (third status) presumed by the presumption unit 705.

Accordingly, with the present embodiment, it is possible to acquire biological information values including the “respiratory rate”, the “heart rate”, the “amount of activity”, the “absence-from-bed”, and the “presence-on-bed” and the statuses of the user (first status and second status), and presume a status of the user (third status) from the information.

### 3. Third Embodiment

Subsequently, a third embodiment will be described. In the third embodiment, the functional configuration in FIG. 2 in the first embodiment is replaced with that in FIG. 5.

In addition to the functional configuration in the first embodiment, a diary output unit 650 is further included. In place of the presumption unit 700, a presumption unit 750 that uses the neural network to presume a status of the user is included.

The diary output unit 650 outputs the acquired biological information values and status of the user (0: absence-from-bed, 1: presence-on-bed and wakening, 2: sleeping), as image data (image data of “1440 pixels×pixels for the number of days”) that is a value of a pixel value for every one minute in diary data in which one line is set as 24 hours, as a diary (patient diary) of a patient that is a user. The diary output unit 650 is capable of outputting, as a diary, a respiratory diary expressing the respiratory rate of the user, a heartbeat diary expressing the heart rate of the user, a sleep diary expressing a sleeping status of the user, an activity amount diary expressing body movement of the user, a respiratory event diary expressing the number of respiratory events, a periodic body movement diary expressing the number of periodic body movement events, and the like. The diary output unit 650 may output these parameters being combined as one diary. The diary output unit 650 is capable of outputting a graph of these diaries as diary data that is image data.

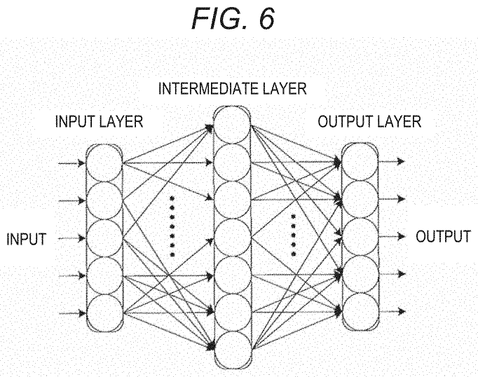

The presumption unit 750 presumes a status of the user (third status) from the inputted diary data. As processing of presuming a status of the user, a deep learning method (deep neural network) recently has a high accuracy specially in image recognition, and the presumption unit 750 uses the method. Processing in this deep learning will be simply described using FIG. 6.

Firstly, the presumption unit 750 inputs a signal of diary data (image data) that the diary output unit 650 outputs into a neural network including a plurality of layers and neurons included in each layer. Each neuron receives signals from the plurality of other neurons, and outputs the signals subjected to the computation to the plurality of other neurons. When the neural network has a multilayer structure, the layers are called an input layer, an intermediate layer (hidden layer), and an output layer through which signals flow in this order.

A neural network including an intermediate layer of a plurality of layers is called a deep neural network (for example, a convolutional neural network having convolution computations), and a method of machine learning using the deep neural network is called deep learning.

The presumption unit 750 subjects neurons of each layer in the neural network to various kinds of computations (convolution computation, pooling computation, normalization computation, matrix computation, and the like), the neurons flowing while changing the shape, and outputs a plurality of signals from the output layer. The presumption unit 750 uses a learned model for presuming a status (third status) of the user from the diary data that is image data, and determines the structure and the parameter of the neural network. The presumption unit 750 then inputs diary data into the neural network based on the learned model, thereby outputting a plurality of signals from the output layer. The learned model may be prepared in advance, or the presumption unit 750 may learn data in which diary data and information on the status of the user are paired as teacher data, thereby generating the learned model.

The presumption unit 750 refers to a plurality of output values from the neural network, the output values respectively being associated with statuses of patients, and presumes the status of the patient who is associated with the largest output value. If the status of the patient is not directly output from the neural network, the presumption unit 750 may cause one or a plurality of output values to pass through a classifier, and may presume a status of the patient from an output from the classifier.

As parameters that are coefficients to be used in various kinds of computation in the neural network, a large number pieces of diary data and statuses of patients corresponding to the diary data are input into the neural network in advance. The parameter is determined in such a manner that an error between the output value and the correct value is propagated in the neural network in the reverse direction by Backpropagation, and the parameter of the neuron in each layer is updated many times. A process of updating and determining the parameter in this manner is called learning.

The structure of the neural network and the individual computations are publicly known techniques that already have been explained in books and papers, and any one of these techniques may be used.

The presumption unit 750 is used to presume a status of the user (third status) from the input parameters such as the biological information values of the user and the statuses of the user (first status and second status).

The presumption unit 750 uses the neural network on the basis of the diary data in which one line is set as 24 hours in the above-mentioned embodiment, and may be based on other diary data. For example, the presumption unit 750 may use diary data in which one line is set 7 days by considering the rhythmicity on a week unit, diary data in which one line is set 28 days by considering the rhythmicity on an approximate month unit, and diary data in which one line is set 365 days by considering the rhythmicity on a year unit. The presumption unit 750 may input biological information values without considering the rhythmicity in advance, and may use the neural network. The presumption unit 750 may presume a status of the user in such a manner that information such as the “heart rate”, the “respiratory rate”, and the “amount of activity”, the “absence-from-bed” and the “presence-on-bed” is input into the neural network by being synchronized to each time axis, and is to be learned. The case where the presumption unit 750 uses the neural network to presume a status of the user has been explained in the present embodiment, but the presumption unit 750 may naturally use a method of machine learning other than the neural network. For example, as a method of machine learning, the presumption unit 750 may use support vector machine, decision tree, or Naive Bayes. The presumption unit 750 may generate a learned model in accordance with a machine learning to be used, based on the teacher data.

### 4. Fourth Embodiment

Subsequently, a fourth embodiment will be described. A case where the presumption unit 700 uses neural network will be described in the fourth embodiment, which is different from the first embodiment.

The presumption unit 700 uses the above-mentioned various parameters. For example, in the present embodiment, the presumption unit 700 uses the “respiratory rate”, the “heart rate”, and the “amount of activity” calculated from the body vibration data acquired by the first acquisition unit 200. The presumption unit 700 also can use the “respiratory event index” and the “periodic body movement index” calculated from the same body vibration data. The presumption unit 700 also can use the status of the user (first status) determined from the determination unit 350 and the status of the user (second status) acquired from the second acquisition unit 600, in other words, the statuses of the user such as “absence-from-bed”, “presence-on-bed”, “sleeping”, and “wakening” of the user.

The presumption unit 700 inputs these biological information values and the statuses of the user (hereinafter, referred to as “user information”) into a neural network including a plurality of layers and neurons included in each layer. Each neuron receives signals from the plurality of other neurons, and outputs the signals subjected to the computation to the plurality of other neurons. When the neural network has a multilayer structure, the layers are called an input layer, an intermediate layer (hidden layer), and an output layer through which signals flow in this order. The neural network has been explained in the other embodiment, and details thereof are omitted. The presumption unit 700 presumes a status of the user (third status) from the user information in this manner.

The presumption unit 700 may use recurrent neural network (RNN) in a case of time-series data such as biological information value calculated based on the biological signal and the sleeping status. This aims to use time-series data by expanding the above-mentioned method of neural network. Examples of recurrent neural network include various networks such as Elman network, Jordan network, Echo State Network, and Long Short-Term Memory network (LSTM). The presumption unit 700 is capable of appropriately presuming a status of the use by using a suitable network.

For example, in Elman network, not only data at a time t, but also data in the hidden layer (intermediate layer) at a time t−1 can be used. Such network configuration allows past biological information on the user to have an influence on a current prediction. The presumption unit 700 is capable of presuming a status of the user (third status) also on the basis of the relationship in time.

With the present embodiment, it is possible to appropriately presume a status of the user from various user information (for example, the “respiratory rate”, the “heart rate”, the “sleeping status (REM sleep, non-REM sleep, shallow sleep, deep sleep, and the like)”, the “amount of activity”, the “fluctuation of respiratory”, the “fluctuation of heartbeat”, the “respiratory event index”, the “periodic body movement index”, “sleeping”, “wakening”, “presence-on-bed”, “absence-from-bed”, and the like), using the neural network, the recurrent network, or the like. The present embodiment may be implemented by a method of machine learning that can use time-series data other than the neural network and the recurrent network. The presumption unit 700 may generate a learned model using a pair of the user information and the status of the user as teacher data.

### 5. Example

An example that uses the above-mentioned embodiments to presume a status of a user will be described.

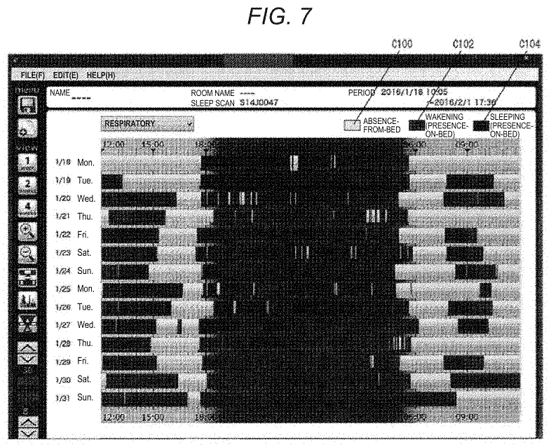

FIG. 7 is a diagram for explaining one example of a diary. The diagram illustrates one example of a sleep diary in which the sleeping status of the user is expressed daily. In the graph in FIG. 7, graphs indicating a sleeping status daily are illustrated in the vertical direction. For example, absence-from-bed may be illustrated in white (C100), wakening (presence-on-bed) in orange (C102), and sleeping (presence-on-bed) as blue (C104). In FIG. 7, the absence-from-bed, the wakening (presence-on-bed), and the sleeping (presence-on-bed) are sequentially displayed darker in color.

The biological information value normally indicates the periodical variations, such as 24 hours or one week. Using the diary in this manner causes a health care worker, a staff, and the like to easily see the long-term variation of the user, for example. This brings an advantage that the staff or the like can quickly recognize a poor physical condition of the user. The day-to-day change is displayed by the diary data to enable the staff or the like to presume with high accuracy a status of the user. For example, in a case where during a time zone when the heart rate indicates an abnormality value, the other biological information values such as the respiratory rate indicate no change, a case where an abnormality value is indicated in a constant time zone every day, and other cases, the staff or the like is capable of determining no abnormality or the like as a status of the user.

[5.1 Explanation of Each Diary Data]

A plurality of diaries can be output. A staff or the like uses a diary, and manually and visually recognizes and determines as a status of the user being abnormal. With the system according to the present embodiments, it is possible to automatically presume or report on a status of the user, independent of the differences among individuals and the ability of persons who make determinations, with the constant reference as appropriate and without burden of the visual recognition and determination.

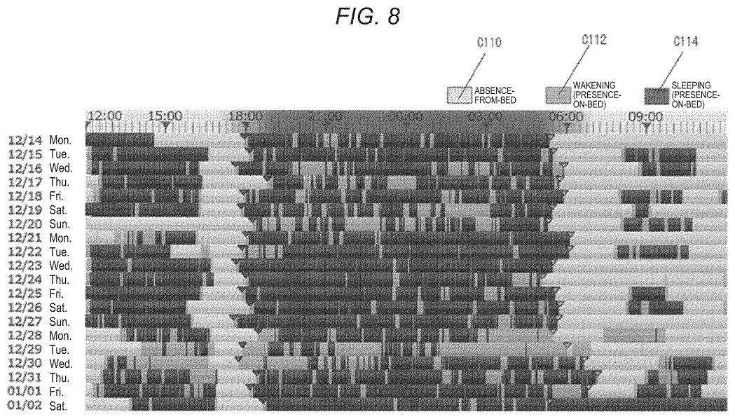

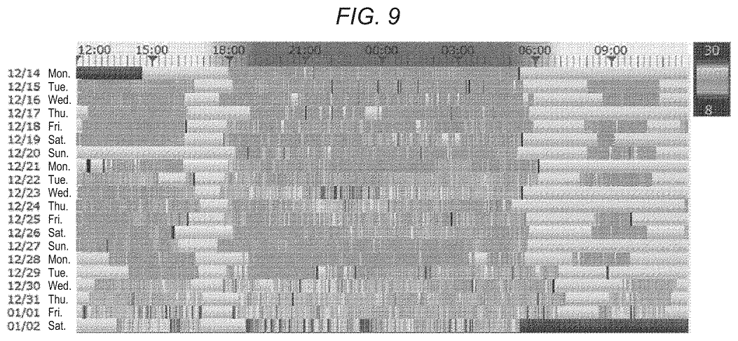

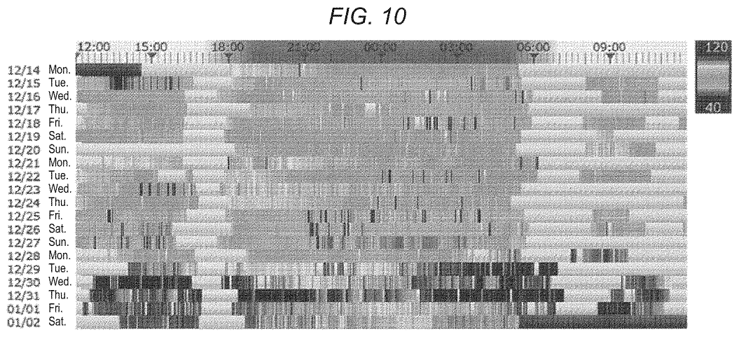

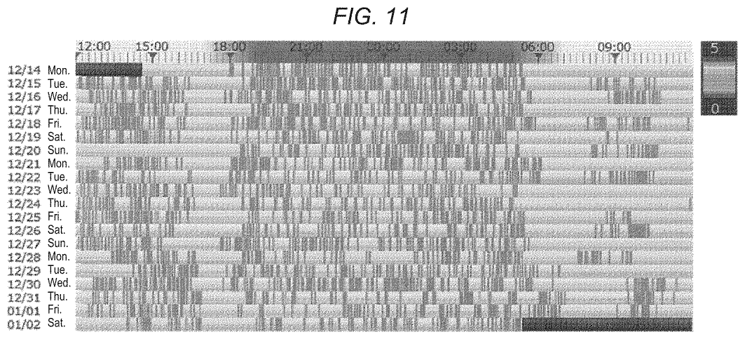

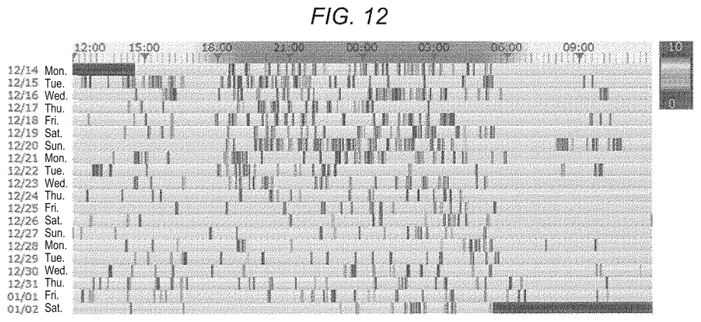

For example, the diary data in FIG. 8 to FIG. 12 illustrate diaries of an elderly person who lives in a certain care facility and needs care before hospitalization. FIG. 8 illustrates a sleep diary, FIG. 9 illustrates a respiratory diary, FIG. 10 illustrates a heartbeat diary, FIG. 11 illustrates a respiratory event diary, and FIG. 12 illustrates a periodic body movement diary.

In all the diaries, one line indicates 24 hours, and the center of the graph indicates 0:00 a.m. This user was hospitalized at about 05:30 a.m. on January 2 (Sat.), and the poor physical condition before the hospitalization can be read from the graph. The change in physical condition can be read from about December 21 to December 23.

The sleep diary in FIG. 8 indicates the time when the user is absent from the bed (almost white, C110 in FIG. 8), the time when the user is present on the bed but is awake (gray/yellow when expressed in color, C112 in FIG. 8), and the time when the user is present on the bed and is sleeping (almost black/blue when expressed in color, C114 in FIG. 8). With reference to a period from about December 21 to December 23, the staff or the like understands that the presence-on-bed time of the user has not changed but the awake status is reduced. Normally, the reduction in the awake status is a good change for the user.

The respiratory diary in FIG. 9 is a graph illustrating the respiratory rate of the user using the colors or the gradation within a range of 8 to 30. For example, when the respiratory rate is expressed in color, it is preferable to express the respiratory rate of the user being 30 or more in red (the high respiratory rate is expressed in color in the red system), and the respiratory rate being 8 or less is expressed in blue (the low respiratory rate is expressed in color of the blue system). When the respiratory diary is expressed in color, the change in the respiratory rate from 8 to 30 stepwisely changes the color as blue→light blue→green→yellow→orange→red. The graph is displayed in a gray scale in FIG. 9.

In the graph on December 23 (Wed.), yellow and red (light gray in FIG. 9) are increased from 18:00 to 04:00, which indicates an abnormality in the respiratory rate of the user. No abnormality of the user is indicated on December 21 (Mon.) and 22 (Tue.).

The heart rate diary in FIG. 10 is a graph illustrating the heart rate of the user using the colors or the gradation within a range of 40 to 120. For example, when the heart rate diary is expressed in color, it is preferable to express the heart rate of the user being 120 or more in red (the high heart rate is expressed in color in the red system), and the heart rate being 40 or less is expressed in blue (the low heart rate is expressed in color of the blue system). When the heart rate diary is expressed in color, the change in heart rate from 40 to 120 stepwisely changes the color as blue→light blue→green→yellow→orange→red. The color changes in 81 stages, for example, in the present example. FIG. 10 illustrates the graph in a gray scale.

For example, in the heart rate diary, almost gray appears (green when expressed in color) until December 20, and light gray (yellow, red, and the like when expressed in color) slightly appears on December 21, and at from 18:00 to 22:00 on December 23. This indicates that the heartbeat of the user is somewhat irregular. In the heart rate diary, almost white (red when expressed in color) appears at 04:00 or later on December 28, which indicates that the condition of the user becomes worse.

The respiratory event diary in FIG. 11 is a graph illustrating the respiratory event index of the user using the colors or the gradation within a range of 0 to 5. For example, when the respiratory event diary is expressed in color, it is preferable to express the respiratory event index of the user being 5 or more in red (the high respiratory event index is expressed in color in the red system), and the respiratory event index of the user being 0 is expressed in blue (the low respiratory event index is expressed in color of the blue system). When the respiratory event diary is expressed in color, the change in respiratory event index from 0 to 5 stepwisely changes the color as blue→light blue→green→yellow→orange→red. The graph is displayed in a gray scale in FIG. 11.

In the respiratory event diary in FIG. 11, the respiratory event index of the user does not indicate the very distinct change. The respiratory event diary in FIG. 11 indicates that the respiratory event index is slightly decreased from about December 22. Normally, the reduction in the respiratory event index is a good change for the user.

The periodic body movement diary in FIG. 12 is a graph illustrating the periodic body movement index of the user using the colors or the gradation within a range of 0 to 10. For example, when the periodic body movement diary is expressed in color, it is preferable to express the periodic body movement index of the user being 10 or more in red (the high periodic body movement index is expressed in color in the red system), and the periodic body movement index of the user being 0 is expressed in blue (the low periodic body movement index is expressed in color of the blue system). The change in periodic body movement index from 0 to 10 stepwisely changes the color as blue→light blue→green→yellow→orange→red. The graph is displayed in a gray scale in FIG. 12.

The periodic body movement diary in FIG. 12 indicates that as a change in periodic body movement index of the user, the periodic body movement is decreased from about December 22. The periodic body movement diary in FIG. 12 indicates that the condition of the user is changed (the reduction in periodic body movement is originally good, however, in the present example, it can be presumed that the condition of the user becomes worse with the change in other biological information value).

In this case, when a status of the user is presumed, with a method of presuming an abnormality only from the respiratory rate and the heart rate, a staff, if can notice earlier, can notice an abnormality only in the morning on December 24, and can reliably notice at night on December 30. On the other hand, when presuming a status of the user by adding statuses of the user (present-in-bed status of the absence-from-bed and the presence-on-bed, a sleeping status, or the amount of activity such as sleeping determined from the amount of activity in FIG. 8), the respiratory event, and the periodic body movement, the system 1 can presume an abnormality on December 22 if early, or on December 24 at the latest. By using a plurality of biological information values, indices, and statuses of the user in this manner, the system 1 can presume a status of the user (third status) in combination thereof.

The system 1 reports on the basis of the presumed status of the user (third status), and thus is capable of presuming the status of the user more appropriately than a case where simply presuming a status of the user (third status) to be abnormal or normal with one parameter (biological information).

[5.2 Fever Case]

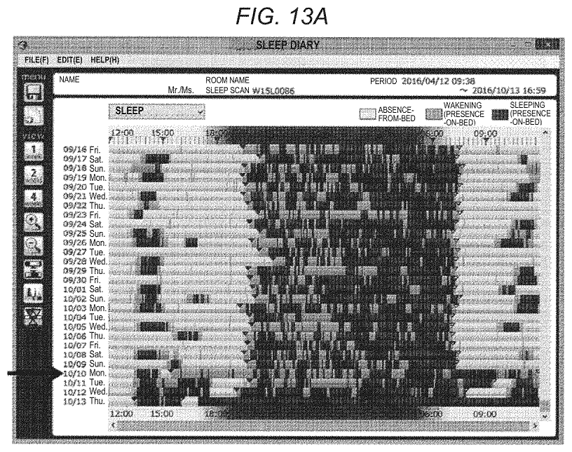

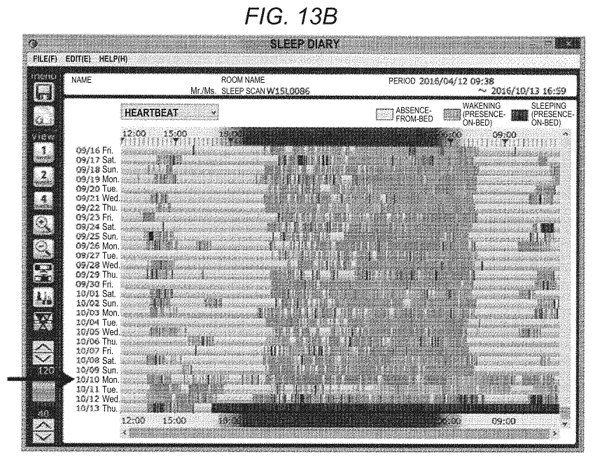

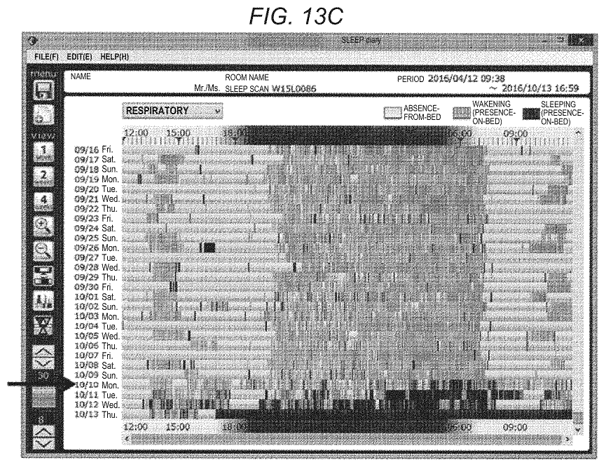

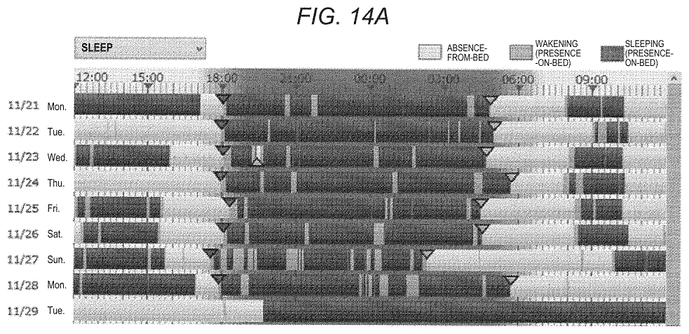

FIGS. 13A to C each are one example of a diary for explaining of a fever case of a user. FIG. 13A illustrates a sleep diary illustrating a sleeping status of the user (the amount of activity may be also used because the sleep was determined from the amount of activity in the present case), FIG. 13B illustrates a heartbeat diary illustrating the heart rate of the user, and FIG. 13C illustrates a respiratory diary illustrating the respiratory rate of the user.

The color is changed depending on a numerical value in the heartbeat diary and the respiratory diary. For example, when these diaries are expressed in color, it is preferable to display an upper limit (for example, the heart rate being 120 or more, the respiratory rate being 30 or more) as red, and a lower limit (for example, the heart rate being 40 or less, the respiratory rate being 8 or less) as blue. When the diaries are expressed in color, for example, the color stepwisely changes from red, orange, yellow, green, light blue, to blue. The staff or the like is capable of grasping the status and the change of the numerical values by seeing these diaries. The diaries in FIGS. 13A to C are displayed in a gray scale. Also in this case, the darkness of the color corresponding to the upper limit or the lower limit may be determined, and the color may be stepwisely changed.

These graphs indicate, for example, that the presence-on-bed time increases on October 10, and the respiratory rate and the heart rate rise from the night to the morning. These graphs indicates that the heart rate of the user is lowered on October 12, but the respiratory rate is high.

With the change in the status of the user illustrated in the drawings, the patient status presumption unit 700/750 presumes a status of the user to be “fever”. The status of the user is determined to be abnormal, and an alert is output (reported). The system 1 combines the statuses of the user (for example, sleep diary), and thus can obtain an effect of estimating an abnormality of the user more surely than a case where an abnormality is determined only from the respiratory rate and the heart rate, and reporting.

[5.3 Pneumonia Case]

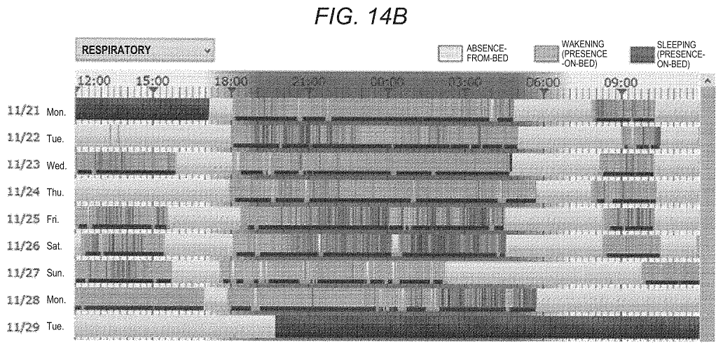

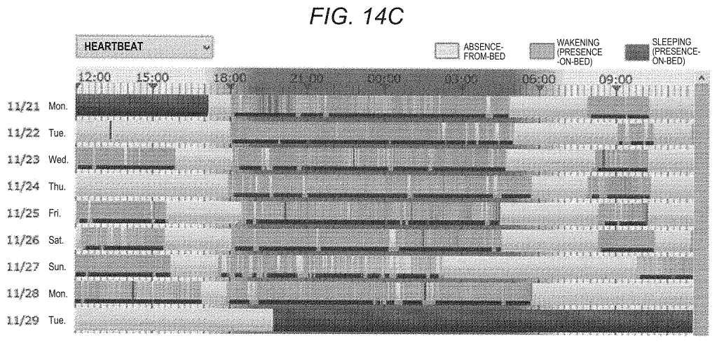

FIGS. 14A to C each are one example of a diary for explaining of a pneumonia case of a user. FIG. 14A illustrates a sleep diary illustrating a sleeping status of the user, FIG. 14B illustrates a respiratory diary illustrating the respiratory rate of the user, and FIG. 14C illustrates a heartbeat diary illustrating the heart rate of the user. In the heartbeat diary and the respiratory diary in FIGS. 14A to C, a graph based on each biological information is displayed in the upper stage (wide region), and a graph based on the sleeping status is displayed in the lower stage (narrow region), for each day.

Firstly, in the sleep diary in FIG. 14A, the time when the user wakes up on November 27 is at about 2:00 a.m., while the user normally wakes up at about 5:00 a.m. This indicates that the user wakes up at the time earlier than usual. In the respiratory diary in FIG. 14B, the graph in green is more increased from at about 18:00 on November 27 than usual, and indicates that the respiratory rate of the user increases. In the heartbeat diary in FIG. 14C, the graph in yellow is increased from at about 0:00 on November 27, and indicates that the heart rate of the user has changed. On the other hand, the respiratory rate and the heart rate of the user do not indicate abnormality values at all.

The system 1 can presume the status of the user to be abnormal and report in the morning on November 28 by referring to the respiratory rate and the heart rate of the user, together with the status of the user (for example, the sleeping status illustrated in the sleep diary). Actually, an abnormality of this patient had been presumed only from the respiratory rate and the heart rate, the abnormality was found by the staff through the medical interview at about 10:00 a.m. on November 29, which is later by one day or more, and this patient was hospitalized due to pneumonia.

For example, when a status of the user is presumed and the respiratory rate and the heart rate are referred, it can be found that slight changes are indicated on points other than November 27. The system 1 reports on the abnormality in all the points to result in many incorrect reports. As indicated in the present embodiment, it is possible to suitably report on an abnormality by presuming a status of the user (third status) using the respective biological information values and the status of the user as parameters.

### 6. Effect

With the above-mentioned embodiments, by using the respiratory rate, the heart rate, and the body movement (amount of activity) measured on the bed during the sleep time (from the time when the user goes to bed to the time when the user wakes up) or during the night (constant time zone such as 23:00 to 5:59), a biological information value highly related to the abnormality is acquired every day under the uniformed condition. It is possible to presume and report an abnormality of the user with high accuracy by using the acquired biological information value and the status of the user.

The system in the above-mentioned embodiments can continuously acquire the respiratory rate, the heart rate, and the body vibration, which are highly related to the status of the user determined as to be abnormal, every day under the uniformed condition, and thus is capable of accurately grasping the change from the data for a prolonged period of time. This enables the system to presume and report on an abnormality of the user without being affected by differences among individuals and the measurement error. The body movement occurs even during the sleep time or during the night. The system includes the body movement in the analysis item (input parameter), which allows the presumption and the report of the abnormality by taking the variation of the heart rate and the respiratory rate due to the body movement artifact and the noise, and the lowering of the accuracy into consideration.

### 7. Modification Example

Although the embodiments of the present invention have been described in details with reference to the drawings, the specific configuration is not limited to the embodiments, and the designs and the like that do not depart from the gist of the present invention are included in the scope of the present invention.

In the present embodiment, the processor 5 outputs biological information on the basis of the result output by the detection device 3, however, the detection device 3 may calculate all the result. The present invention may be implemented not only by being an application installed to the terminal device (for example, a smartphone, a tablet computer, and a computer), but also, for example, processing the data at the server side, and returning the process result to the terminal device.

For example, the detection device 3 uploads biological information to the server, and the above-mentioned process may be implemented on the server side. The detection device 3 may be implemented, for example, a device such as a smartphone in which an acceleration sensor and a vibration sensor are incorporated.

The program that operates in the respective devices in the embodiments is a program (program that causes the computer to function) that controls the CPU and the like so as to implement the functions in the above-mentioned embodiments. The information that is handled in these devices is temporarily accumulated in a temporary storage (for example, RAM) when being processed, is thereafter stored in various kinds of storages such as a ROM, an HDD, or an SSD, and is read, corrected, and written by the CPU if necessary.

When the program is distributed on the market, the program can be stored in a transportable recording medium and distributed, or may be transferred to a server computer that is connected via a network such as the Internet. In this case, a storage in the server computer is naturally included in the present invention.

### REFERENCE SIGNS LIST

1 abnormality reporting system
3 detection device
5 processor
100 control unit
200 biological signal acquisition unit
300 biological information value calculation unit
350 sleeping status determination unit
400 input unit
450 output unit
500 memory
510 biological information data
520 sleeping status data
600 patient status acquisition unit
650 patient diary output unit
700, 705, 750 patient status presumption unit
710 feature extraction unit
720 identification unit
730 identification dictionary
740 patient status output unit
800 alert output unit
10 bed
20 mattress

1-13. (canceled)

14. A device comprising:
a sensor configured to acquire a biological signal of a user on a bed;
a controller configured to presume a status of the user only around a first timing every day on the basis of the biological signal, the first timing being a timing the user wakes up; and
a output unit configured to output an alarm when the status of the user is abnormal.

15. The device according to claim 14, wherein the controller is configured to calculates a heart rate and a respiratory rate of the user from the biological signal, and the controller is configured to presume the status of the user on the basis of the heart rate or the respiratory rate of the user.

16. The device according to claim 14, wherein the controller is configured to calculates a heart rate, a respiratory rate, an amount of activity of the user, whether the user is on the bed or not, a respiratory event index and a periodic body movement index from the biological signal, and the controller is configured to presume the status of the user on the basis of at least the two of the heart rate, the respiratory rate, and the amount of activity, whether the user is on the bed or not, a respiratory event index and a periodic body movement index.

17. The device according to claim 14, wherein the controller is configured to calculate a biological information value from the biological signal, and the controller is configured to presume the status of the user by weighting the biological information value in accordance with a type of the sensor.

18. The device according to claim 16, wherein the controller is configured to calculate a biological information value from the biological signal, and the controller is configured to presume the status of the user by weighting the biological information value in accordance with a type of the sensor.

19. The device according to claim 14, further comprising:
a memory configured to store the biological signals of at least one week, and wherein the sensor is provided between the bed and a mattress on the bed.

20. The device according to claim 19,
Wherein output unit is configured to output a belt-like graph expressed daily in accordance with the biological signals stored in the memory for at least plurality of days, and
wherein the controller is configured to presume the status of the user on the basis of the belt-like graph.

21. The device according to claim 14, further comprising:
a generator configured to generate a model for presuming the status of the user by machine learning using the biological signal, and
wherein the controller is configured to presume the status of the user by inputting the biological signals into the model.

22. The device according to claim 14, wherein the controller is configured to determine the status of the user is abnormal if a plurality of requirements are satisfied, the controller is configured to determine the status of the user is normal even if one of the requirements is satisfied, and the requirement is a requirement related to the biological signal to judge the status of the user is abnormal.

23. The device according to claim 22, wherein the requirement correspond to one of the following parameters is abnormal,
(1) an average respiratory rate for a latest period,
(2) a difference between the latest and past average values of the average respiratory rate during the night,
(3) an average heart rate for a latest period,
(4) a difference between the latest and past average values of the average heart rate during the night,
(5) a fluctuation of the respiratory rate during the night,
(6) a fluctuation of heart rate during the night,
(7) a difference between the latest and past average values of the average amount of activity during the night,
(8) an average amount of activity for the latest period.

24. A reporting method in a device capable of reporting an abnormality if a status of a user being abnormal, the device including a sensor, a controller, and a output unit, the reporting method comprising:
an acquisition step of acquiring a biological signal of the user on a bed, by the sensor;
a presumption step of presuming a status of the user only around a first timing every day on the basis of the biological signal, by the controller; and
a report step of reporting an alarm when the status of the user is abnormal at the presumption step, by the output device.
wherein the first timing being a timing the user wakes up.

25. The reporting method according to claim 24, further comprising
a calculating step of calculating a heart rate and a respiratory rate of the user from the biological signal, and
wherein the a presumption step of presuming the status of the user on the basis of the heart rate or the respiratory rate of the user.

26. The reporting method according to claim 24, further comprising
a calculating step of calculating a heart rate, a respiratory rate, an amount of activity of the user, whether the user is on the bed or not, a respiratory event index and a periodic body movement index from the biological signal, and
wherein the a presumption step of presuming the status of the user on the basis of at least the two of the heart rate, the respiratory rate, and the amount of activity, whether the user is on the bed or not, a respiratory event index and a periodic body movement index.

27. The reporting method according to claim 24, further comprising
a calculating step of calculating a biological information value from the biological signal, and
wherein the a presumption step of presuming the status of the user by weighting the biological information value in accordance with a type of the sensor.

28. The reporting method according to claim 26,
wherein the calculating step including a step of calculating a biological information value from the biological signal, and the controller is configured to presume the status of the user by weighting the biological information value in accordance with a type of the sensor.

29. The reporting method according to claim 24, further comprising:
a storing step of storing the biological signals of at least one week in a memory, and wherein the sensor is provided between the bed and a mattress on the bed.

30. The reporting method according to claim 29,
wherein the report step of outputting a belt-like graph expressed daily in accordance with the biological signals stored in the memory for at least plurality of days, and
wherein the a presumption step of presuming the status of the user on the basis of the belt-like graph.

31. The reporting method according to claim 24,
wherein the a presumption step including a step of determining the status of the user is abnormal if a plurality of requirements are satisfied by the controller,
wherein the a presumption step including a step of determining the status of the user is normal even if one of the requirements is satisfied, and the requirement is a requirement related to the biological signal to judge the status of the user is abnormal.

32. The reporting method according to claim 24, wherein the requirement correspond to one of the following parameters is abnormal,
(1) an average respiratory rate for a latest period,
(2) a difference between the latest and past average values of the average respiratory rate during the night,
(3) an average heart rate for a latest period,
(4) a difference between the latest and past average values of the average heart rate during the night,
(5) a fluctuation of the respiratory rate during the night,
(6) a fluctuation of heart rate during the night,
(7) a difference between the latest and past average values of the average amount of activity during the night,
(8) an average amount of activity for the latest period.
